(19) United States
(12) Patent Application Publication
(10) Pub. No.: US 2008/0292616 A1
(43) Pub. Date: Nov. 27, 2008

(54) Topical Formulations of Histone Deacetylase Inhibitors and Methods Using the Same

(75) Inventors: Susan E. Bates, Bethesda, MD (US); Antonio Tito Fojo, Rockville, MD (US); Richard L. Piekarz, Silver Spring, MD (US); John J. Wright, Falls Church, VA (US); George J. Grimes, Rockville, MD (US); Karen M. Schweikart, Centreville, VA (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: Government of the United of America, Represented by the Secretary, Department of Health and...., Rockville, MD (US)

(21) Appl. No.: 12/064,220

(22) PCT Filed: Aug. 15, 2006
(86) PCT No.: PCT/US06/31870

§ 371 Date: Aug. 15, 2008

Related U.S. Application Data
(60) Provisional application No. 60/709,553, filed on Aug. 19, 2005.

(51) Int. Cl. A61K 38/02; A61K 39/395; A61K 38/07; A61K 31/165; A61P 35/00

(52) U.S. Cl. 424/130.1; 514/2; 514/18; 514/646

(57) ABSTRACT

Disclosed are topical compositions comprising at least one histone deacetylase (HDAC) inhibitor (HDI) and a carrier comprising petrolatum. Methods for using such compositions to treat or inhibit cancer and various skin diseases are also disclosed.

### BACKGROUND OF THE INVENTION

Histone deacetylase inhibitors (HDIs) are a class of antineoplastic agents that have been the subject of some clinical trials. HDIs induce growth arrest usually associated with cellular differentiation or apoptosis. Alterations in the enzymes controlling histone acetylation and deacetylation have been shown to be a direct transformation mechanism is some malignancies. One particular HDI is the depsipeptide FK228, e.g., stereoisomer FR901228.

Cutaneous T cell lymphoma (CTCL) is a rare form of lymphoma that has a great unmet need for effective, low toxicity therapies. CTCL is an indolent disorder of malignant, relatively mature T-cells which frequently involves the skin, bloodstream, regional lymph nodes and spleen. Approximately 800-1,000 new cases are diagnosed per year in the U.S. There are several clinical variants of the disease. The condition causes severe skin itching, pain and edema. Patients with advanced disease have extensive involvement of their skin with lymphoma resulting in disfigurement and often skin pain and itching. CTCL patients can be affected by skin tumors, skin ulcers, as well as repeated shedding of the skin. While there are treatments for the disease, the disease's progression is often impossible to stop. With the loss or deterioration of the barrier normally provided by the skin, CTCL patients usually die from infections.

Clinical trials with internal administration of FR901228 to treat CTCL have been performed but have limitations in respect to toxicity and effectiveness. There exists a need to identify alternative modes of administration for FR901228 and other HDIs to help reduce toxicity and increase effectiveness of HDIs in treating CTCL, other cancers, as well as other diseases. The present invention provides such an alternative mode of administration.

### BRIEF SUMMARY OF THE INVENTION

The invention provides topical compositions comprising at least one histone deacetylase (HDAC) inhibitor (HDI) and a carrier comprising petrolatum. In one aspect, the invention provides a composition suitable for topical application, the composition comprising one or more HDI selected from the group consisting of a peptide, an antibody, an antigen binding fragment of an antibody, a nucleic acid, an aliphatic acid, a hydroxamic acid, a benzamide, depudecin, and an electrophilic ketone, and a salt, a prodrug, and a combination thereof; and a topical carrier comprising petrolatum. In another aspect, the invention provides a composition suitable for topical application, the composition comprising a HDI or a prodrug, or a salt thereof, wherein the HDI is a depsipeptide; and a topical carrier comprising petrolatum.

The invention also provides methods of using one or more of the topical histone deacetylase inhibitor (HDI) compositions of the invention to treat cancer and various skin diseases, as well as molecules and physiological processes associated with the same. In one aspect, the invention provides a method for treating or inhibiting cancer comprising topically administering an effective amount of a topical HDI composition. In another aspect, the invention provides a method of reducing the number of T cells in the skin comprising topically administering an effective amount of a topical HDI composition. In another aspect, the invention provides a method for treating or inhibiting an immunological skin disorder comprising topically administering an effective amount of a topical HDI composition. Such immunological skin disorders include without limitation cutaneous manifestation of lupus, drug eruption, contact dermatitis, and a combination thereof.

### DETAILED DESCRIPTION OF THE INVENTION

In one aspect, the invention provides a composition suitable for topical application, the composition comprising one or more histone deacetylase (HDAC) inhibitors (HDIs) selected from the group consisting of a peptide, an antibody, an antigen binding fragment of an antibody, a nucleic acid, an aliphatic acid, a hydroxamic acid, a benzamide, depudecin, and an electrophilic ketone, and a salt, a prodrug, and a combination thereof; and a topical carrier comprising petrolatum. In another aspect, the invention comprises a composition suitable for topical application, the composition comprising a HDI or a prodrug, or a salt thereof, wherein the inhibitor is not a butyrate salt; and a topical carrier comprising petrolatum. In another aspect, the invention provides a composition suitable for topical application, the composition comprising a HDI or a prodrug, or a salt thereof, wherein the HDI is a depsipeptide; and a topical carrier comprising petrolatum. In some embodiments, the topical carrier comprising petrolatum further comprises at least one component selected from the group consisting of mineral oil, ceresin, and lanolin alcohol. In some embodiments, the topical carrier comprising petrolatum further comprises at least one component selected from the group consisting of panthenol, glycerin, and bisabolol.

Histone deacetylases represent a class of enzymes, also called protein deacetylases, that catalyze removal of an acetyl group from the epsilon-amino group of lysine side chains in histones or other proteins (for example, histones H2A, H2B, H3 or H4), thereby reconstituting a positive charge on the lysine side chain. Several histone deacetylases have been identified, including, but not limited to HDAC1, HDAC2, and RPD3. Specific, non-limiting examples of a histone deacetylase include, but are not limited to, GenBank Accession Nos. NM 058277, NM15401, AF407273, XM 004379, and AF 426160, AF006603, AF006602, and AF074882; see also U.S. Pat. No. 6,287,843. When histone deacetylase is inhibited, the activity of the counter enzyme, histone acetyltransferase, is in relative excess, and hyperacetylation of histones or other proteins occurs. Without being bound by theory, inhibition of histone deacetylase results in the lysine tails of histones becoming neutralized, disruption of the histone structure, and unfolding of DNA. The unfolded state of the histone permits transcription factors to access the DNA.

A histone deacetylase inhibitor (HDI) is an agent that inhibits the function of one or more histone deacetylases, for example, by 10%, 20%, 30%, 40%, 50%, 80%, 95% or more. Such agents may take the form of a pharmaceutical agent or drug, a therapeutically effective oligonucleotide, a specific binding agent, or a fragment or variant of histone deacetylase. Several structural classes of histone deacetylase inhibitors including but not limited to (1) short-chain fatty acids, (2) hydroxamic acids, (3) cyclic tetrapeptides containing 2-amino-8-oxo-9,10-epoxy-decanoyl moiety, and (4) benzamides. Specific, non-limiting examples of a histone deacetylase inhibitor include FR901228, N-acetyldinaline (CI-994), Scriptaid, suberoylanilide hydroxamic acid, trichostatin A, trapoxin A, trapoxin B, HC-toxin, chlamydocin, Cly-2, WF-3161, Tan-1746, apicidin, analogs of apicidin, benzamide, derivatives of benzamide, hydroxyamic acid derivatives, azelaic bishydroxyamic acid, actetate salts, suberoylanilide hydroxyamide acid, suberic bishydroxyamic acid, m-carboxy-cinnamic acid bishydroxyamic acid, oxamflatin, depudecin, or MS-275. Alternatively, the agent may be a therapeutically effective oligonucleotide that inhibits expression or function of histone deacetylase, such as an antisense molecule or a ribozyme. Alternatively, a histone deacetylase inhibitor can be a dominant negative fragment or variant of histone deacetylase. Further examples of HDIs are described herein.

HDI can be a peptide, for example, a cyclic peptide, and more particularly a depsipeptide. Depsipeptides are cyclic peptides comprising peptide bonds in a ring structure. The depsipeptide can be a cyclic tetrapeptide. In some embodiments, the depsipeptide is a bicyclic tetrapeptide. In some embodiments, the HDI is a depsipeptide and is more specifically FK228 represented by Formula I:

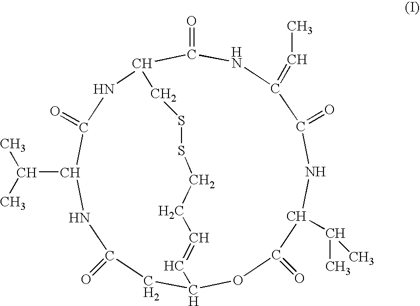

The HDI can be a particular stereoisomer of FK228, e.g., FR901228, which is (E)-(1S, 4S, 10S, 21R)-7-[(Z)-ethylidene]-4,21-diisopropyl-2-oxa-12,13-dithia-5,8,20,23-tetraazabicyclo [8,7,6]-tricos-16-ene-3,6,19,22-pentanone (NSC 630176); see Formula II:

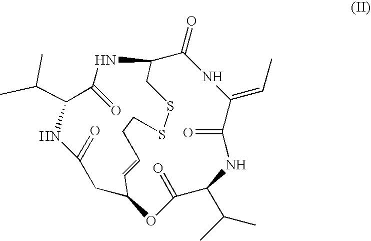

Unless otherwise specified, a simple reference to FK228 in the present specification means a group of compounds, irrespective of the stereoisomerism, including the compound of Formula (II).

FK228 and a salt thereof are known substances and are obtainable by various means such as bacterial production, synthethic, and semi-synthetic means. For example, FR901228, which is one of the stereoisomers of FK228, can be obtained by culturing, under aerobic conditions, a bacterial strain belonging to the genus Cromobacterium, e.g., Cromobacterium violaceum WB968 (FERM BP-1968), see, e.g., JP-B-7-64872. Various FK228 stereoisomers can be produced according to the method reported by Khan W. Li, et al., J. Am. Chem. Soc., 118:7237-7238 (1996).

The salt of a HDI, e.g., FK228, is a biologically acceptable salt, which is generally non-toxic, and is exemplified by salts with base or acid addition salts, inclusive of salts with inorganic base such as alkali metal salt (e.g., a sodium salt, a potassium salt, etc.), alkaline earth metal salt (e.g., calcium salt, magnesium salt, etc.), ammonium salt, salts with organic base such as organic amine salt (e.g., triethylamine salt, diisopropylethylamine salt, pyridine salt, picoline salt, ethanolamine salt, diethanolamine salt, triethanolamine salt, dicyclohexylamine salt, N,N′-dibenzylethylenediamine salt, etc.), inorganic acid salt (e.g., hydrochloride, hydrobromide, sulfate, phosphate, etc.), organic carboxylic or sulfonic acid salt (e.g., formate, acetate, trifluoroacetate, maleate, tartrate, fumarate, methanesulfonate, benzenesulfonate, toulenesulfonate, etc.), salt with basic or acid amino acid (e.g., arginine, aspartic acid, glutamic acid, etc.), and the like.

The present invention includes within its scope prodrugs of the HDIs. Such prodrugs can be functional derivatives of the HDIs that are readily convertible in vivo into a particular HDI. Prodrugs can be selected from various covalently bonded groups such as esters and amides. Examples of prodrugs include esters, acetates, formates, benzoate derivatives of alcohols, amides, and amines. They also include derivatives of the amidine or guanine functionality and would include C(═NR3)NH2 where R3 is selected from OH, NH2, C1-4 alkoxy, C6-10 aryloxy, C1-10 alkoxycarbonyl, C6-10 aryloxycarbonyl. Preferred derivatives include examples wherein R3 is OH, NH2, methoxy, and ethoxycarbonyl. Conventional procedures for the selection and preparation of suitable prodrug derivatives are described, for example, in Design of Prodrugs, ed. H. Bundgaard, Elsevier (1985).

An HDI, e.g., FK228, can have stereoisomers based on asymmetric carbon atoms and double bonds, such as optical isomers, geometric isomers, and the like, all of which and mixtures thereof are also encompassed in the present invention. Further solvate compounds (e.g., inclusion compounds such as hydrate, etc.) of HDIs and salts thereof are also encompassed by the present invention.

In addition to or in the alternative to FK228, the HDI can be or include a cyclic tetrapeptide such as apidicin, FR225497, trapoxin A, chlamydocin, didemnin B, CHAP, HC-toxin, WF27082, sandramycin, and a combination thereof.

In some embodiments, the HDI can be or include an aliphatic acid or a salt thereof such as valproic acid, valeric acid, isovaleric acid, propionic acid, 3-bromopropionic acid, butyric acid, 2-propylpentanoic acid, and a combination thereof. In some embodiments, the HDI can be or include a hydroxamic acid. Examples of hydroxamic acids include trichostatin A (TSA), trichostatin C, salicylihydroxamic acid (SBHA), azelaic bishydroxamic acid (ABHA), azelaic-1-hydroxamate-9-anilide (AAHA), 6-(3-chlorophenylureido) carpoic hydroxamic acid (3Cl-UCHA), oxamflatin, A-161906, Scriptaid, PXD-101, MW2796, MW2996, suberoylanilide hydroxamic acid (SAHA), LAQ824, m-carboxylcinnamic acid bishydroxamate (CBHA), CHAP, and pyroxamide, and a combination thereof.

In some embodiments, the HDI can be or include depudecin or a prodrug thereof. In some embodiments, the HDI can be or include an electrophilic ketone. Examples of electrophilic ketones include trifluoromethylketone and α-keto amides. An example of an α-keto amide is N-methyl-α-ketoamide. In some embodiments, the HDI can be or include a benzamide. Examples of benzamides without limitation are MS-27-275 (MS-275), a 3′-amino derivative of MS-27-275, and CI-994, and a combination thereof.

In some embodiments, the HDI of the topical composition is a specific binding agent such as an antibody or a polypeptide comprising an antigen binding fragment thereof that binds a histone deacetylase so that the enzyme is inhibited. A specific binding agent is an agent that specifically binds only to a defined target. Thus a histone deacetylase-specific binding agent binds substantially only a histone deacetylase such as a particular histone deacetylyase isoform. Anti-histone deacetylase protein antibodies can be produced using standard procedures described in a number of texts, including Harlow and Lane, Antibodies, A Laboratory Manual, CSHL, New York (1988). The determination that a particular agent binds substantially only to a histone deacetylase may readily be made by using or adapting routine procedures. One suitable in vitro assay makes use of the Western blotting procedure (described in many standard texts, including Harlow and Lane, Antibodies, A Laboratory Manual, CSHL, New York (1988); Ausubel et al., in Molecular Biology, CSHL, New York (1998).

Shorter fragments of antibodies can also serve as specific binding agents. For instance, Fabs, Fvs, and single-chain Fvs (SCFvs) that bind to a histone deacetylase are histone deacetylase-specific binding agents. These antibody fragments are defined as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)₂, the fragment of the antibody obtained by treating whole antibody with the enzyme pepsin without subsequent reduction; (4) F(ab′)2, a dimer of two Fab′ fragments held together by two disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule.

In some embodiments the HDI of the topical composition comprises a nucleic acid molecule. Therapeutically effective oligonucleotides and oligonucleotide analogs are characterized by their ability to inhibit a function of a protein, for example by inhibiting the expression of a protein. Inhibition can be any reduction in target protein activity or expression seen when compared to target protein activity or expression in the absence of the oligonucleotide or oligonucleotide analog. Additionally, some oligonucleotides will be capable of inhibiting the activity or expression of a target protein by at least 15%, 30%, 40%, 50%, 60%, or 70%, or more.

Some therapeutically effective oligonucleotides and oligonucleotide analogs are additionally characterized by being sufficiently complementary to target protein-encoding nucleic acid sequences. As described herein, sufficiently complementary means that the therapeutically effective oligonucleotide or oligonucleotide analog can specifically disrupt the expression of the target protein, and not significantly alter the expression of genes other the target nucleic acid sequence. For example, a therapeutically effective oligonucleotide can reduce histone deacetylase activity in a cell by at least 15%, 30%, 40%, 50%, 60%, or 70%, or more.

The term “oligonucleotide” refers to an oligomer or polymer of ribonucleic acid or deoxyribonucleic acid. This term includes oligonucleotides composed of naturally-occurring nucleobases, sugars and covalent intersugar (backbone) linkages as well as oligonucleotides having non-naturally-occurring portions that function similarly. Such modified or substituted oligonucleotides are often preferred over native forms because of desirable properties such as, for example, enhanced cellular uptake, enhanced binding to target and increased stability in the presence of nucleases.

The compositions can be provided in the form of a unit dose, wherein the HDI and topical carrier are not in admixture but are packaged together for co-administration. The compositions can also be used for the manufacture in a medicament for treating various conditions and diseases including without limitation those described herein.

The petrolatum comprising topical carrier may comprise different amounts of petrolatum. For example, the petrolatum can be in an amount from about 1 to about 99%, e.g., 5 to 90%, such as about 10%, 20%, 30%, 40%, 50%, 60%, 70%, 80%, or 90% of the topical carrier by weight. In some embodiments, the petrolatum is 41% of the carrier by weight. Petrolatum is a purified mixture of semisolid hydrocarbons, chiefly of the methane series of the general formula CₙH₂ₙ₊₂. Petrolatum is also characterized as a colloidal system of nonstraight-chain solid hydrocarbons and high-boiling liquid hydrocarbons in which most of the liquid hydrocarbons are held inside micelles. For more information on petrolatum see Schindler, Drug Cosmet. Ind. 89:36-37, 76, 78-80, and 82 (1961). The topical carrier may further comprise one or more additional components, e.g., mineral oil, ceresin, lanolin alcohol, panthenol, glycerin, bisabolol, cocoa butter, water, mineral oil, isopropyl myristate, PEG-40 sorbitan peroleate, glyceryl lanolate, sorbitol, propylene glycol, cetyl palmitate, magnesium sulfate, aluminum stearate, BHT, methylchloroisothiazolinone, and methylisothiazolinone. Each of these further components can be present in a suitable amount, e.g., from about 0.1 to about 99% or from about 0.01 to about 50%, e.g., 0.1-20% or 1-10%. Suitable compositions comprising petrolatum and one or more further components include, for example, Aquaphor® and Eucerin®. The HDI can be incorporated into the topical carrier in any suitable amount. For example, the HDI can be present in at least about 0.001%, 0.01%, 0.05%, 0.1%, 0.2%, 0.3%, 0.4%, 0.5%, 0.6%, 0.7%, 0.8%, 0.9%, 1.0%, 1.5%, 2%, 2.5%, 3%, 4%, or 5% by weight.

The invention also provides methods using one or more of the topical histone deacetylase inhibitor (HDI) compositions of the invention to treat cancer and various skin diseases, as well as cells, pathways, systems, and physiological processes associated with the same. The methods can be practiced on any type of animal. In some embodiments, the animal is a mammal such as rodent or a primate. Examples of rodents include mice, rats, and rabbits. An example of a primate is a human.

The invention provides a method for treating or inhibiting cancer comprising topically administering an effective amount of a topical HDI composition. The treatment or inhibition is applicable to any suitable cancer, e.g., a lymphoma, such as a postthymic lymphoma. For example, the postthymic lymphoma can be a T cell lymphoma. T cell lymphomas are comprised of a spectrum of clinical phenotypes, ranging from low-grade, cutaneous T cell lymphomas (CTCL) to highly aggressive peripheral T cell lymphoma (PTCL). Some PTCLs are termed “unspecified.” CTCL is a form of non-Hodgkin's lymphoma of helper T-cells that arises in the skin and can be manifested as Mycosis Fungoides, Sézary Syndrome, as well as intermediate manifestations. In some embodiments, the T cell lymphoma is a peripheral T cell lymphoma. In some embodiments, the T cell lymphoma is a cutaneous T cell lymphoma. In some embodiments, the cancer is a B cell lymphoma.

The invention provides a method of reducing the number of T cells in the skin, blood, and/or adjoining tissue comprising topically administering an effective amount of a topical HDI composition. In some embodiments, this reduction comprises a reduction in the number of malignant T cells. In some embodiments, the reduction comprises a reduction in the number of helper T cells. Reduction in T cell numbers, malignant or otherwise, can be associated with treatment of cancer as well as various skin diseases.

The invention provides a method for treating or inhibiting an immunological skin disorder comprising topically administering an effective amount of a HDI composition. Such immunological skin disorders include cutaneous manifestation of lupus, drug eruption, contact dermatitis and a combination thereof.

By “effective amount” is meant an amount of histone deacetylase inhibitor sufficient to treat cutaneous T-cell lymphoma in the mammal, in particular a human, over a reasonable time frame. The determination of an effective amount is within the ordinary skill in the art. The dose administered to a mammal, particularly a human, in the context of the present invention will vary with the inhibitor administered (e.g., its potency), the composition employed, the route of administration, the severity of the disease state, the body weight, sex, and age of the subject, the extent of contact, and the particular site being treated. The size of the dose also can be determined by the existence of any adverse side effects that can accompany the use of the particular inhibitor employed. Adverse side effects are kept to a minimum where possible.

The topical compositions of the present invention are administered using a dosage regime effective to treat or inhibit a given condition or disease. Generally, when an inhibitor is administered to an animal, such as a mammal, in particular a human, the inhibitor can be administered in a dose of from about 1 to about 1,000 micrograms or about 1 to about 1,000 milligrams of the inhibitor per kg of the body weight of the host per day. However, this dosage range is merely exemplary, and higher or lower doses may be chosen in appropriate circumstances. For instance, the actual dose and schedule can vary depending on whether the composition is administered in combination with one or more other therapies such as radiation or pharmaceutical compositions, or depending on interindividual differences in pharmacokinetics, drug disposition, and metabolism. For example, the composition can be administered at a dose of about 1.25 or about 2.5 mg/kg/d of the HDI. 2.5 mg/kg/day can be equivalently administered as 30 mg/m²/d. Any appropriate conversion between mg/kg/d and mg/kg/day can be utilized as understood by those of ordinary skill in the art. An exemplary conversion is a 70-75 kg individual with a 2 m² body surface area. Other exemplary doses include at least about 0.01, 0.05, 0.1, 0.5, 0.75, 1, 1.5, 1.75, 2.0, 2.25, 2.75, 3.0, 3.25, 3.5, 3.75, 4.0, 4.25, 4.5, 4.75, 5.0, 7.5, 10, 12.5, 15, 20, 25, 30, 40, 50, 100, 250, 500, and 750 mg/kg/d of the HDI. In some embodiments, the dose can be between about 2.25 to about 2.75, about 2 to about 3, about 1.5 to about 3.5, about 1 to about 4, about 0.5 to about 4.5, about 0.1 to about 10, about 0.05 to about 25, or about 0.01 to about 750 mg/kg/d.

In some embodiments, the HDI topical composition is administered daily, for example, the composition can be administered for 14 days, or for 28 days (4 weeks). The composition can be administered for a fraction of a day or the entire day, e.g., for 1 hr., 2 hrs., 3 hrs., 4 hrs., 6 hrs., 8 hrs., 12 hrs., 16 hrs., 20 hrs., 24 hrs., or any period in between. In some embodiments, the composition is administered 1, 2, 3, 4, 5, 6, or 7 times a week. In some embodiments, the composition is administered until a Draize score of about 0 to about 1.9, about 2.0 to about 5.0, or about some intermediate value thereof is obtained. For example, the tolerable schedule for depsipeptide administration is days 1, 8, and 15 of a 28 day cycle, such as at a 14 mg/m² dose or days 1 and 5 of a 21 day cycle, such as at a 18 mg/m² dose.

The topical composition can be administered to a target area of skin of any desired area, e.g., about 2 sq. in. to about 20 sq. in. Examples include 2 sq in., 5 sq. in., 10 sq. in., 15 sq. in., and 20 sq. in. In some embodiments, two 10 sq. in. patches are used. In some embodiments, the topical composition is administered to between 1 and 4 target areas, and in some, 5 and 10 target areas. In some embodiments, the topical composition is applied to 10%, 25%, or 50% of body surface area. In some embodiments, the topical composition is administered to at least about 10%, at least about 25%, or at least 50% of body surface area. The topical composition can be administered to part of, substantially all of, or the entire body surface area. The topical composition can be applied to any portion of the body surface.

In some embodiments, the composition is administered topically as well as by one or more additional routes of administration including but not limited to systemic administration. If the histone deacetylase inhibitor is systemically administered, preferably it is administered orally or by intravenous infusion. Compositions suitable for oral and intravenous infusion are also known in the art. The compositions can be applied using a skin patch delivery system. Such a patch includes a reservoir such as a matrix for the composition, a mechanism that allows the composition to be released at a desired rate, and optionally a mode for attachment to the skin.

Formulations suitable for oral administration can consist of (a) liquid solutions, such as an effective amount of the compound dissolved in diluent, such as water, saline, or orange juice; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth. Pastilles can comprise the active ingredient in an inert base, such as gelatin and glycerin, or sucrose and acacia, emulsions, gels, and the like containing, in addition to the active ingredient, such excipients/carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

When the histone deacetylase inhibitor is administered systemically, preferably the administration is intermittent. For example, if the histone deacetylase inhibitor NSC 630176 is administered by intravenous infusion, it can be administered at the maximum tolerated dose of 17.8 mg/m2 over 4 hours on days 1 and 5 of a 21-day cycle, for example, although other doses and schedules can be effective and can be determined in accordance with methods known in the art. Certain HDIs, such as FR901228, can cause toxicity problems when present in the bloodstream above certain levels. Accordingly, in some embodiments, it is desirable to limit the amount of HDI in the bloodstream. However, in certain conditions such as Sezary's Syndrome, the cancerous cells can be present in the bloodstream as well. Accordingly, depending on the particular cancer, one can vary the means and extent of the administration. For example, when applied topically, the topical composition can be applied to only disease affected areas and/or to non-disease affected areas of the body surface.

The methods of the invention can include treatment with one or more HDIs as well as combinations of one or more HDI with another therapeutic. In some embodiments, a method further comprises (i) administering a steroid, a P-glycoprotein multiple drug resistance (MDR) antagonist, a retinoid and/or an antibody to a T-cell receptor, (ii) the use of chemotherapy, (iii) the use of photochemotherapy, and/or the use of radiotherapy.

Examples of steroids that are suitable for use in the context of the present invention include, but are not limited to, glucocorticoids. Preferably, a steroid is administered topically. P-glycoprotein antagonists are also known in the art and include, but are not limited to, cyclosporin A, verapamil, quinidine, dihydro-pyridines, calcium channel blockers, cyclosporin analogues (e.g., PSC833 (Novartis, East Hanover, N.J.)), phenothiazines, thioxanthenes, XR9576 (Xenova, Flough, United Kingdom), GG918 (Glaxo), VX710 (Vertex), and others of similar or greater potency. Preferably, a P-glycoprotein antagonist is administered topically or systemically. Retinoids include agents that bind to the retinoic acid receptor, such as 9-cis-retinoic acid, 4-hydroxy-retinoic acid, all trans-retinoic acid, (E)-4-[2-(5,6,7,8-tetrahydro-2-naphthylenyl)-1-propenyl]-benzoic acid, and 3-methyl-(E)-4-[2-(5,6,7,8-tetrahydro-2-naphthylenyl)-1-propenyl]-benzoic acid). A retinoid is preferably administered topically or systemically. A commercially available antibody to a T-cell receptor is Zenapax, which is available from Hoffman-LaRoche, Inc., Nutley, N.J. The antibody, or antibody binding fragment thereof, is preferably administered systemically.

To monitor the effectiveness, toxicity, and other parameters, various tests and assays can be performed before, during, and/or after the administration of the compositions of the invention. Examples of such tests include biopsies, pharmacokinetic assays, and immunohistochemical analyses. Biopsies can be carried out in a number of different ways. For example, skin and tumor biopsies are evaluated in order to determine changes in mRNA expression of depsipeptide target genes, e.g., changes in the level of induction of p21, cyclin E and/or MDR-1. In some embodiments, one looks for increased expression. Immunohistochemical analyses can include those that measure levels of histone acetylation. A subject receiving one or more of the topical compositions of the invention who experiences a negative reaction can be given a rest period before resumption of the therapy. Dosage and route of administration can be varied during a period of administration of one or more of the compositions of the invention.

The following examples further illustrate the invention but, of course, should not be construed as in any way limiting its scope.

### EXAMPLE 1

This example demonstrates an advantage of the topical composition in accordance with an embodiment of the invention. A study uses New Zealand white rabbits to determine topical toxicity of the depsipeptide FR901228 when applied topically in either Cetaphil° lotion (CL) or Aquaphor cream (AC). The Cetaphil lotion, which does not comprise petrolatum, contains purified water, glycerin, hydrogenated polyisobutene, cetearyl alcohol and ceteareth-20, macadamia nut oil, dimethicone, tocopheryl acetate, stearoxytrimethylsilane (and) stearyl alcohol, panthenol, farnesol, benzyl alcohol, phenoxyethanol, acrylates/cl 0-30 alkyl acrylate crosspolymer, sodium hydroxide, and citric acid. 201 mg of FR901228 (Fujisawa) was 40 g Cetaphil (Galderma) is macerated with a glass mortar for 20 minutes or until all is incorporated. The finished composition is placed in ointment jars. The Aquaphor cream comprises petrolatum (41%) as well as mineral oil, ceresin, and lanolin alcohol. 216 mg of FR901228 (depsipeptide powder equivalent to pure peptide) and 40 g of Aquaphor ointment (cream) (Beiersdorf, Inc.) are mixed in a glass mortar until homogeneous. The finished product is placed in tubes. Both the AC and CL compositions should generally have a shelf life of 1.5 years under refrigeration (e.g., 2-8° C.).

The dose of depsipeptide is 2.5 mg/kg/day (30 mg/m2/d); controls receive vehicle (Aquaphor or Cetaphil) in place of depsipeptide. A highly intense dosing regimen is employed to study toxicity. The study is designed for application of the respective topical composition to the rabbits for 4 hours daily for 28 days. The four-hour application is accomplished by removing the applied composition approximately four hours after application. Four groups of rabbits (2 rabbits/sex/group) are used with two control and two experimental groups. The control groups receive carrier alone (CL or AC) and the experimental groups receive FR901228 in the respective carriers. The experimental parameters include weekly testing of clinical pathology and plasma drug levels. Histopathology is performed at days 15 and 28.

Application of the topical compositions are terminated after 14 days due to skin lesions at the application site in the case of both the Cetaphil and the Aquaphor depsipeptide formulations. The rabbits that receive depsipeptide in CL experience edema, discolored and hardened skin, as well as sloughing. The depsipeptide CL rabbits have a Draize score of 1.5-2.5 indicating a slight to moderate irritant. The rabbits that receive depsipeptide in AC experienced erythema, blistering, and swelling. The depsipeptide AC rabbits have a Draize score of 0.5-1.0 indicating a slight to well-defined irritant. Rabbits in the experimental groups recover so that by day 22 their skin appeared fairly normal. Rabbits in the control (vehicle only) have no observable skin toxicity.

Pharmacology data are collected including mean plasma levels of depsipeptide of 0.78 ng/mL and 0.12 ng/mL on day 4 in rabbits given depsipeptide in the CL and AC formulations respectively. Pharmacodynamic measurements indicate a mild to moderate decrease in red blood cell (RBC) count in both depsipeptide treated groups at both day 8 and day 15. Pathology studies include microscopic lesions by day 15. For the group receiving depsipeptide in CL, mild ulcers and edema is observed along with marked scab formation. For the group receiving depsipeptide in AC, mild suppurative inflammation, mild hemorrhage and necrosis, moderate ulcer, and marked scab formation are observed. Most lesions are resolved by day 28.

The study allows for a number of conclusions. A dose of 2.5 mg/kg depsispeptide (FR901228) administered topically for 14 days to rabbits results in significant skin toxicity at the application site. However, the toxicity appears to be reversible. Depsipeptide in CL appears to cause more observable skin toxicity than with the AC formulation.

### EXAMPLE 2

This example demonstrates that a HDI topical composition in accordance with an embodiment of the invention is well tolerated. The dosing schedule is less intensive than that described in Example 1. The dose used is again 2.5/mg/kg/d (30 mg/m2/d) FR901228 or vehicle in AC, but is applied topically, 4 hrs. a day, for 4 weeks, three times weekly. The study parameters include weekly clinical pathology and histopathology takes place at days 27 and 41. The compositions are applied to substantially all of the rabbit's back. In the case of a human patient or other diseased subject, the composition can be applied to disease-affected and/or non-affected surface areas.

The results show that both the depsipeptide and vehicle control rabbits have red skin at the application site, although onset varies at day 9 and day 17 respectively. Draize scoring in the depsipeptide group is slight to well-defined. Pharmacodynamics shows increased FBN (1.6-2×) on days 4, 11, and 27 in depsipeptide-treated animals. Pathology shows microscopic lesions. At day 27 in the depsipeptide group, ulcers of minimal severity at the application site are observed.

This study shows that 2.5 mg/1 g (30 mg/m2) of FR901228 in AC is well tolerated when administered topically to rabbits three times a week for four weeks.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition suitable for topical application, the composition comprising:
(a) one or more histone deacetylase (HDAC) inhibitors (HDI) selected from the group consisting of a peptide, an antibody, an antigen binding fragment of an antibody, a nucleic acid, or salt thereof, an aliphatic acid, salt, or ester thereof, a hydroxamic acid, a benzamide, depudecin, an electrophilic ketone, a prodrug thereof, and a combination thereof; and
(b) a topical carrier comprising petrolatum.

2. A composition suitable for topical application, the composition comprising:
(a) a histone deacetylase (HDAC) inhibitor (HDI) or a prodrug, or a salt thereof, wherein the inhibitor is not a butyrate salt; and
(b) a topical carrier comprising petrolatum.

3. A composition suitable for topical application, the composition comprising:
(a) a histone deacetylase (HDAC) inhibitor (HDI) or a prodrug, or a salt thereof, wherein the HDI is a depsipeptide; and
(b) a topical carrier comprising petrolatum.

4. The composition of claim 1, wherein the carrier further comprises at least one component selected from the group consisting of mineral oil, ceresin, and lanolin alcohol.

5. The composition of claim 1, wherein the carrier further comprises at least one component selected from the group consisting of panthenol, glycerin, and bisabolol.

6. The composition of claim 1, wherein the HDI peptide is a cyclic tetrapeptide.

7. The composition of claim 6 wherein the cyclic tetrapeptide is a member selected from the group consisting of apidicin, FR901228, FR225497, trapoxin A, chlamydocin, didemnin B, CHAP, HC-toxin, WF27082, sandramycin, and a combination thereof.

8. The composition of claim 1, wherein the HDI is a depsipeptide.

9. The composition of claim 8 wherein the depsipeptide is a bicyclic depsipeptide.

10. The composition of claim 8 wherein the depsipeptide is (E)-(1S, 4S, 10S, 21R)-7-[(Z)-ethylidene]-4,21-diisopropyl-2-oxa-12,13-dithia-5,8,20,23-tetraazabicyclo [8,7,6]-tricos-16-ene-3,6,19,22-pentanone (NSC 630176).

11. The composition of claim 8 wherein the depsipeptide is FR901228 or FK228.

12. (canceled)

13. The composition of claim 1, wherein the HDI is an aliphatic acid selected from the group consisting of valproic acid, valeric acid, isovaleric acid, propionic acid, 3-bromopropionic acid, and a combination thereof or a salt thereof.

14. The composition of claim 1, wherein the HDI is a hydroxamic acid.

15. The composition of claim 14 wherein the HDI is a hydroxamic acid selected from the group consisting of trichostatin A (TSA), trichostatin C, salicylihydroxamic acid (SBHA), azelaic bishydroxamic acid (ABHA), azelaic-1-hydroxamate-9-anilide (AAHA), 6-(3-chlorophenylureido) carpoic hydroxamic acid (3 Cl-UCHA), oxamflatin, A-161906, Scriptaid, PXD-101, MW2796, MW2996, suberoylanilide hydroxamic acid (SAHA), LAQ824, m-carboxylcinnamic acid bishydroxamate (CBHA), CHAP, and pyroxamide, and a combination thereof.

16. The composition of claim 1, wherein the HDI inhibitor is depudecin or a prodrug thereof.

17. The composition of claim 1, wherein the HDI inhibitor is an electrophilic ketone.

18. The composition of claim 17, wherein the electrophilic ketone is selected from the group consisting of a trifluoromethylketone and an α-keto amide.

19. The composition of claim 18, wherein the α-keto amide is N-methyl-α-ketoamide.

20. The composition of claim 1, wherein the HDI inhibitor is a benzamide.

21. The composition of claim 20, wherein the benzamide is selected from the group consisting of MS-27-275 (MS-275), a 3′-amino derivative of MS-27-275, and CI-994, and a combination thereof.

22. A method for treating or inhibiting cancer in a mammal comprising topically administering to the mammal an effective amount of the composition of claim 1.

23. A method of reducing the number of T cells in the skin or adjoining tissue of a mammal comprising topically administering to the mammal an effective amount of the composition of claim 1.

24. The method of claim 23, wherein the reduction is in the number of malignant T cells.

25. The method of claim 23, wherein the reduction is in the number of helper T cells.

26. A method for treating or inhibiting an immunological skin disorder in a mammal comprising topically administering to the mammal an effective amount of the composition of claim 1.

27. The method of claim 26 wherein the immunological skin disorder is selected from the group consisting of a cutaneous manifestation of lupus, a drug eruption, contact dermatitis and a combination thereof.

28-43. (canceled)

44. The method of claim 22 wherein the mammal is a human.

45-52. (canceled)

53. The method according to claim 22, wherein the cancer is a lymphoma.

54-62. (canceled)
